(19) United States
(12) Patent Application Publication
(10) Pub. No.: US 2009/0018581 A1
(43) Pub. Date: Jan. 15, 2009

(54) SOFT TISSUE FIXATION ASSEMBLY AND METHOD

(75) Inventors: Justin Anderson, Edwards, CO (US); Paul Westhaver, Dartmouth (CA); Paul V. Fenton, JR., Marblehead, MA (US)

Correspondence Address: FOLEY & LARDNER LLP, 111 HUNTINGTON AVENUE, 26TH FLOOR, BOSTON, MA, 02199-7610, US

(73) Assignee: Axya Medical, Inc.

(21) Appl. No.: 11/776,009

(22) Filed: Jul. 11, 2007

(51) Int. Cl. A61B 17/04

(52) U.S. Cl. 606/232

(57) ABSTRACT

A soft tissue fixation assembly comprises an anchor element which is installed in a bone or other tissue and a suture joiner element which mates with the anchor element. The suture joiner element includes a suture retaining element that securely retains suture and which is connected to the anchor element. Energy is transmitted through the suture joiner element to cause relative vibratory motion between the respective components and localized collapsing and compressing of the suture joiner element to secure the suture within the suture joiner element and to secure the suture joiner element within the anchor element. The soft tissue segment is thus fixed to the bone via the sutures secured in the bone anchor.

### FIELD OF THE INVENTION

The invention is related to surgical fixation devices for fixing soft tissue to bone, and in particular to soft tissue fixation devices that include a bone anchor and suture.

### BACKGROUND OF THE INVENTION

In the surgical repair of soft tissue, such as, for example, the surgical reattachment of a torn ligament to bone, it is known to use multi-part devices to fix the soft tissue to the bone. The multi-part devices typically include a screw or other bone anchoring device, and a button-like device for anchoring the suture therein. The anchor is installed in a predrilled hole in the bone, and the soft tissue is fixed to the anchor in the bone with sutures, which are fastened together with the button instead of with knots.

A disadvantage of such devices is that the quality and strength of the device may be limited by the quality and strength of the suture, and/or by the integrity of the attachment of the device to the bone. The soft tissue will detach from the anchor in the bone if the suture slips or breaks. If the anchor or the button slips or becomes dislodged, the soft tissue will not remain anchored to the bone.

U.S. Pat. No. 6,056,751 (Fenton) attempts to address this problem of slipped suture by providing a sutureless soft tissue fixation assembly. However, that invention may not work well in situations where the configuration of the tissue does not allow proper alignment of the tissue relative to the anchor. There remains a need for a suture device and method for securing soft tissue, such as labrum tissue.

It would therefore be an advantage to provide a surgical soft tissue fixation device which overcomes the disadvantages of the prior art devices.

### SUMMARY OF THE INVENTION

The invention provides an integrated soft tissue fixation assembly which attaches securely soft tissue segments to bone. The assembly includes two pieces which are fused together in situ in a patient, which allows for securely holding tissue in place with the use of sutures.

According to one aspect of the invention, the assembly comprises a bone anchor element, adapted for installation into a hole in a bone and including an anchor portion at a distal end and a drive portion at a proximal end, and a suture joiner element. The anchor element and the joiner element are adapted to be joined together in situ. The joiner element, in one embodiment, includes a suture retaining element at or near the proximal end thereof, which suture retaining element includes a groove extending at least partially circumferentially thereabout for receiving suture.

The anchor element preferably includes threads or barbs at its distal end for effecting a substantially permanent installation of the anchor portion into a bone. The anchor element is adapted for mating engagement with the joiner element and includes a radially extending hub and, in one embodiment, at least one bore for receiving a corresponding connection element of the joiner element. One or both of the anchor element and the joiner element includes one or more energy directors for focusing energy applied to either component of the assembly.

The energy directors preferably focus ultrasonic energy applied to the assembly and comprise a plurality of protruding elements that extend outwardly from one or both components. In addition, the suture joiner element is adapted to fuse to or around suture positioned in the groove upon application of energy to the joiner element.

According to another embodiment, the joiner element includes a bore for receiving a complementary pin or leg of the anchor element. At least one of the bore of the joiner element and the anchor element includes one or more energy directors for focusing energy applied to the assembly.

According to another aspect of the invention there is provided a method of fixing soft tissue to a bone. The method comprises the steps of:

providing a soft tissue fixation assembly as described above for attachment of soft tissue to bone;

drilling a hole into a bone at a desired location for installation of the anchor element therein;

installing the anchor element into the drilled hole;

securing suture that is fixed to tissue in the joiner element;

assembling the joiner element into the anchor element through the segment of soft tissue to hold the soft tissue segment within the tissue capture region; and

bonding the joiner element to the anchor element, thereby fixing the soft tissue segment to the bone.

These and other features of the invention will be more fully appreciated with reference to the following detailed description which is to be read in conjunction with the attached drawings.

### BRIEF DESCRIPTION OF THE DRAWINGS

The invention is further described by the following description and figures, in which:

FIGS. 1A and 1B are perspective views of embodiments of the soft tissue fixation assembly of the present invention;

FIG. 2 is a perspective view of an embodiment of the suture joiner element of the present invention;

FIG. 3 is a perspective view of an embodiment of the soft tissue fixation assembly of the present invention;

FIG. 4 is a perspective view of an alternate embodiment of the soft tissue fixation assembly of the invention;

FIG. 5 is a partial sectional and partial perspective view of the assembly, showing the pre-drilling step of the claimed method;

FIG. 6 is a partial sectional and partial perspective view of the assembly of FIG. 1B, showing the bone anchor positioning step of the claimed method;

FIG. 7 is a partial sectional and partial perspective view of the assembly of FIG. 1B, showing the bone anchor element in position in the target bone, and the suture joiner element moving into position for securing suture thereto;

FIG. 8 is a partial sectional and partial perspective view of the assembly of FIG. 1B, showing the suture positioned in the suture retaining element as the suture joiner element is moved into position;

FIG. 9 is a partial sectional and partial perspective view of the assembly of FIG. 1B, showing the step of connection the suture joiner element to the bone anchor element; and

FIG. 10 is a partial sectional and partial perspective view of the assembly of FIG. 1B, showing the final step of using ultrasonic energy to secure the suture joiner element with the bone anchor element.

Like elements in the respective FIGURES have the same reference numbers.

### DETAILED DESCRIPTION OF THE PREFERRED EMBODIMENT

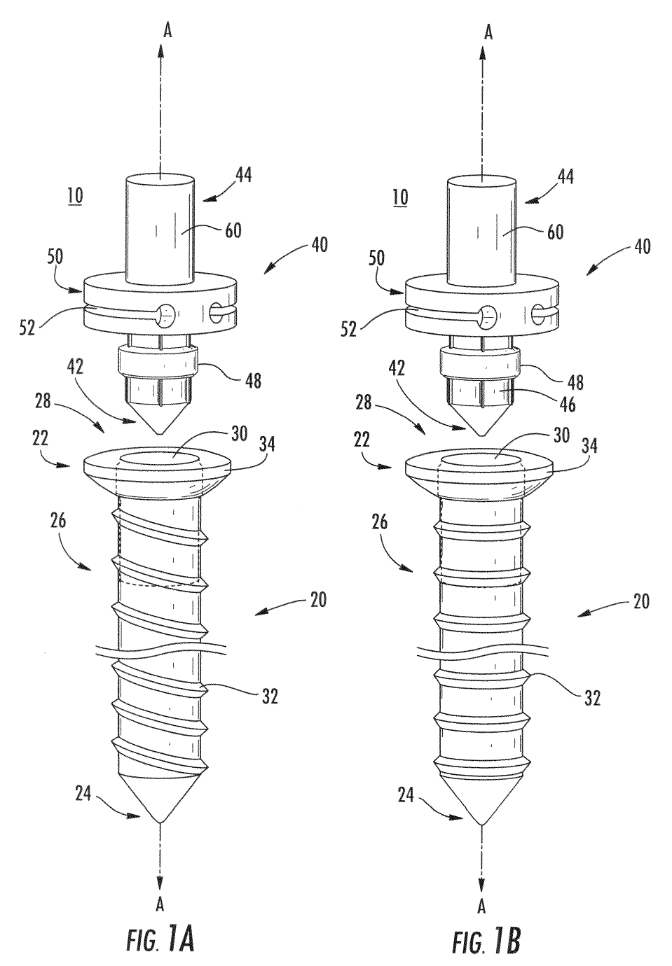

One embodiment of the soft tissue fixation assembly of the present invention is illustrated in FIG. 1A. The assembly 10 comprises an elongated bone anchor element 20, with central bore 30, which extends along an axis A between proximal end 22 and distal end 24. The anchor element 20 includes an anchor portion 26 at its distal end 24 and a drive portion 28 at its proximal end 22. This element 20 is adapted, such as by threads or barbs 32 on the outer surface thereof, to grip a bone into which the anchor element is to be installed. In this embodiment, the anchor portion 26 has a hub 34 at the proximal end 22, which has a diameter greater than the nominal diameter of the anchor portion 26 so that, in one embodiment, the hub 34 acts as a stop for the anchor element 20 during installation into bone.

An alternative embodiment is shown in FIG. 1B. That embodiment is the same as the embodiment of FIG. 1A except that it has concentric rings (or “threads”) 32, instead of a helical thread pattern. The embodiment of FIG. 1B is adapted to be pushed into a hole in a bone, with the rings forming an interference fit.

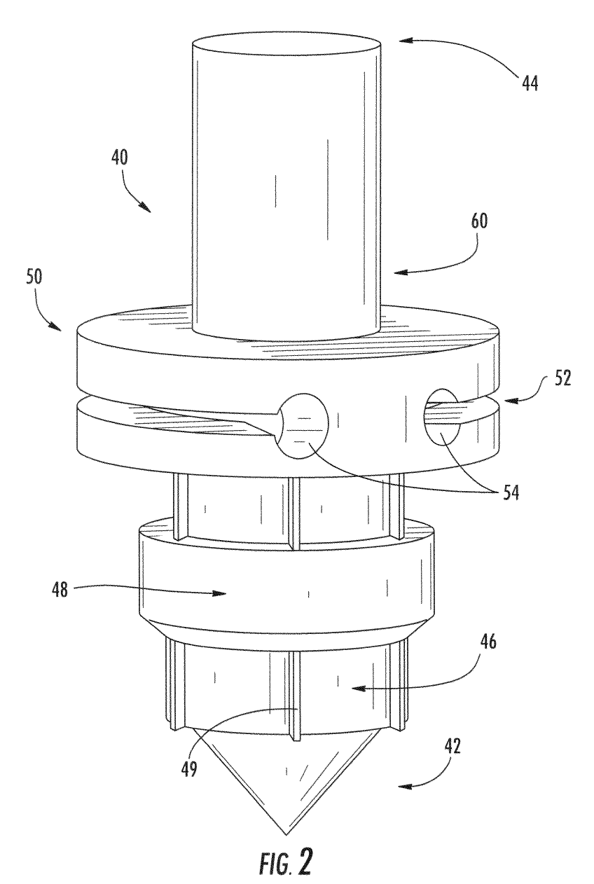

The assembly 10 of FIGS. 1A and 1B further include a suture joiner element 40 which also extends along the axis A between its distal end 42 and its proximal end 44. The suture joiner element 40 is shown in greater detail in FIG. 2. The joiner element 40 includes a suture retaining element 50 extending radially with respect to the axis A. The retaining element 50 includes a groove 52 that extends circumferentially around the retaining element 50 for securing suture therewithin, and as described in further detail below. The nominal diameter of the retaining element 50 preferably is at least equivalent to the diameter of the anchor element 20, depending on the specific configuration of the anchor element, as described further below.

Each of the bone anchor element 20 and the suture joiner element 40 are adapted for substantially permanent attachment to each other. In the illustrated embodiments of FIGS. 1A, 1B, and 2, the suture joiner element 40 includes a joiner connection rod 46 that extends along the axis A from the retaining element 50 toward the distal end 42 of the joiner element 40. The connection rod 46 preferably includes one or more flanges for securing the joiner element 40 to the anchor element 2. In the illustrated embodiment, the flange 48 essentially is a ring of material extending from rod 46 that effects an interference fit (or a snap fit into a recess in anchor portion 26) as the rod 46 is introduced into anchor portion 26. In alternate embodiments, the flange 48 may be multiple pieces of the same materials positioned about or along the connection rod 40. The connection rod 46 also may include one or more energy directors 49 extending radially from the connection rod 46, as shown in FIG. 2. In one embodiment, the flange 48 may function as an energy director, such that the connection rod 46 includes a single structure that functions as a flange to hold the two elements 20, 40 in secure position, and function as an energy director to direct ultrasonic or other energy to connect the two elements 20, 40. Thus, the energy directors 49 may take the form of axial ribs or spines, helical ribs, or threads on the surface of either the connection rod 46 or within the bore 30.

In the illustrated embodiments of FIGS. 1A, 1B, and 2, the suture joiner element 40 includes a drive rod 60 attached to or positioned in the suture retaining element 50. The drive rod 60 is used to insert the suture joiner element 40 in the bone anchor element 20 after the anchor element 20 is positioned in bone, as described in further detail below. In a preferred embodiment, the drive rod 60 is detachably connected to the retaining element 50. In alternate embodiments the drive rod 60 is permanently fixed to the suture retaining element 50, or a single rod that extends through the suture retaining element 50 to form, at its proximal end, the connection rod 46 and at the distal end the drive rod 60.

As shown in FIG. 2, the suture retaining element 50 includes a groove 52 extending at least partially about the circumference of the element. The groove 52 is of sufficient dimensions to accept a suture therein. Elements 50 may have customized grooves to accommodate sutures of different widths and materials, depending on the specific intended use. In the illustrated embodiment, the groove 52 extends partially around the element 50. In alternate embodiments there may be two grooves, each of which extends partially and end-to-end around the circumference of the retaining element 50. In a preferred embodiment, the grooves include a terminal notch 54 which serves to securely hold or lock suture in place once it wraps through the grooves 52. Alternatively, the groove 52 may be a single continuous recess around the circumference of the element 50. The retaining element 50 is manufactured of materials that are formable or that melt upon application of energy such as heat, ultrasonic energy, or other energy, such that application of energy causes the groove to shrink or fuse about suture retained therein. The energy may be directed to the retainer element 50 through energy directors 49 located on the connection rod 46, or by other means as known to those skilled in the relevant art.

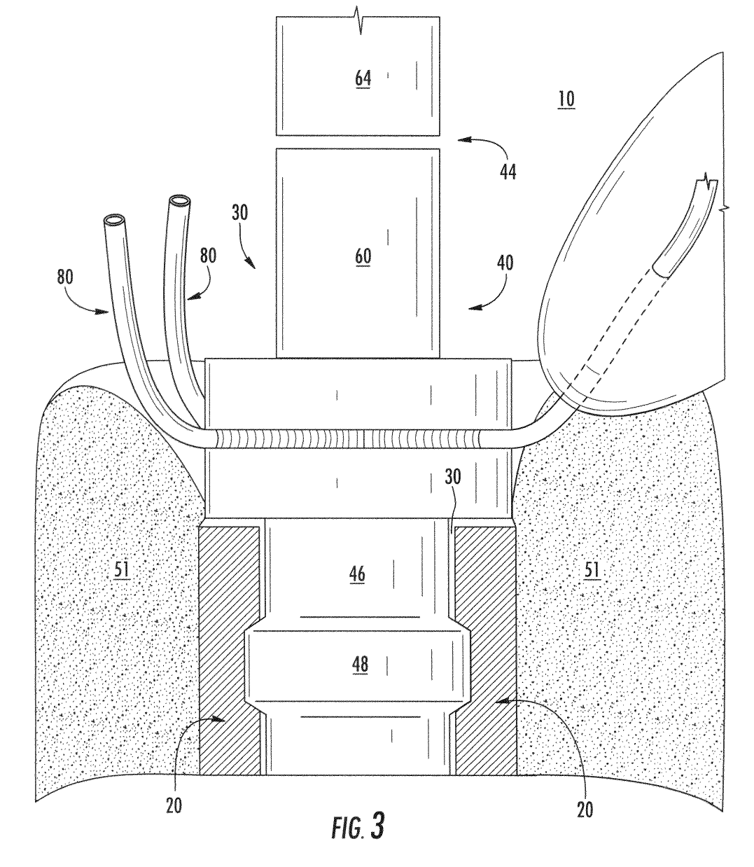

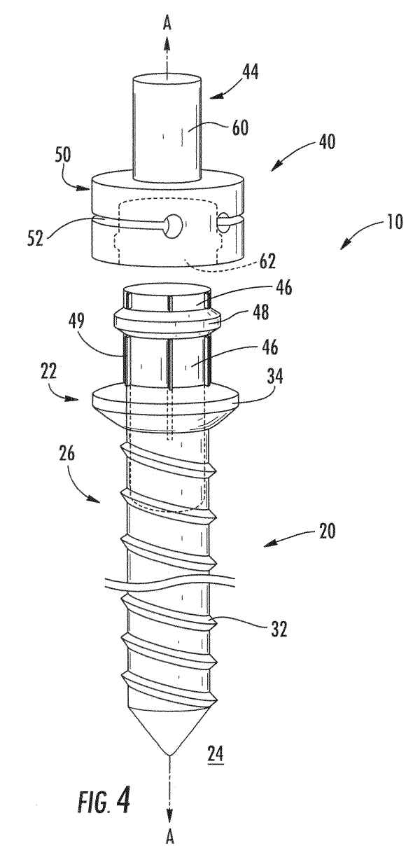

FIG. 3 illustrates the soft tissue fixation assembly of FIGS. 1A and 2 connected and deployed in situ in bone 51. In an alternate embodiment, and as shown in FIG. 4, the connection rod 46 is attached to or integral with the bone anchor element 20 at its distal end. In this embodiment, the connection rod 46 includes a flange 48 and energy directors 49, as described above. In practice, once the anchor element 20 (shown in section in FIG. 3) is securely positioned in bone 51, connection rod 46 is inserted into a joiner bore 62 in suture retaining element 50 where it is held in position by the flange 48. The energy directors 49 then are activated to fuse or otherwise join the connection rod 46 within the joiner bore 62 to fixedly attach the anchor element 20 to the joiner element 40. The ultrasonic welding is performed in situ, once the two elements 20, 40 are securely positioned together.

In both illustrated embodiments, the energy directors 49 focus ultrasonic energy directed into the joiner element 40 or anchor element 20 from an ultrasonic weld horn 64, shown in FIG. 3. The application of this energy establishes an interface between the two elements. In the embodiment of FIGS. 1A and 2, the interface is between the connection rod 46 attached to the suture joiner element 40 and the anchor bore 30, whereas in the embodiment of FIG. 4, the interface is between the anchor rod 46 attached to the bone anchor element 20 and the joiner bore 62. In both embodiments, the effect is to securely connect the bone anchor element 20 and the suture joiner element 40.

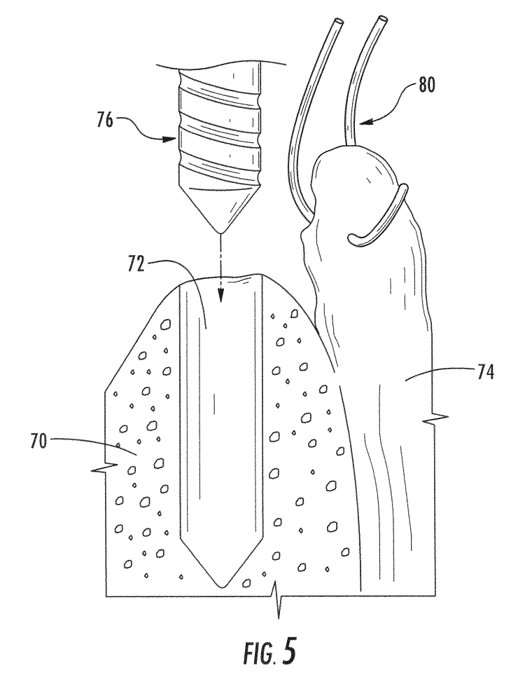

The inventive assembly 10 is shown in practice in FIGS. 5 through 10. The method of using the assembly 10 is substantially the same regardless of which embodiment of bone anchor element 20 or suture joiner element 40 is used. As shown in FIG. 5, a bore 72 is drilled into bone 70, using a bone drill 76 generally known and available to those skilled in the relevant art. Alternatively, the anchor portion 26 of the bone anchor element 20 may include a self-drilling tip so that the anchor element 20 can be driven directly into a bone 70 without predrilling a hole 72.

Also as shown in FIG. 5, suture 80 is drawn through the target soft tissue 74 in a manner specific to the type of tissue, the location, and other factors readily discernible by those surgeons in the relevant art. Thus, a preliminary step includes pre-drilling the anchor hole 72 and securing suture 80 through the target soft tissue 74.

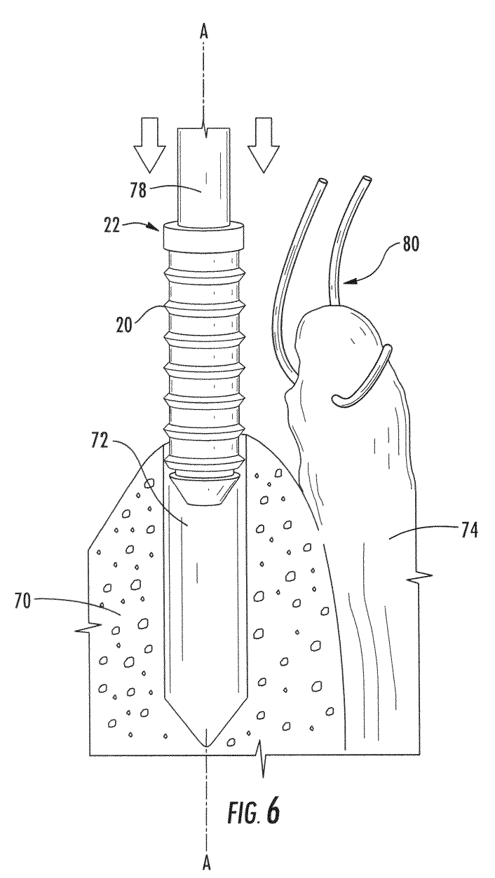

In the illustrated embodiment of FIG. 6, a push rod 78 is positioned against the distal end 22 of the borne anchor element 20, and force is applied thereto to push the bone anchor element 20 into the bone hole 72. The push rod 78 may be positioned within a groove (not shown) at the distal tip of the anchor element 20, or may be screwed into a threaded recess (not shown) at the distal end of the anchor element 20 to prevent the push rod 78 from slipping from position as it pushes the anchor element into the bone hole 72. In the embodiment where the anchor element 20 includes a self-drilling tip, the push rod 78 may be a screwdriver or other element used to drill the anchor element directly into the bone 70.

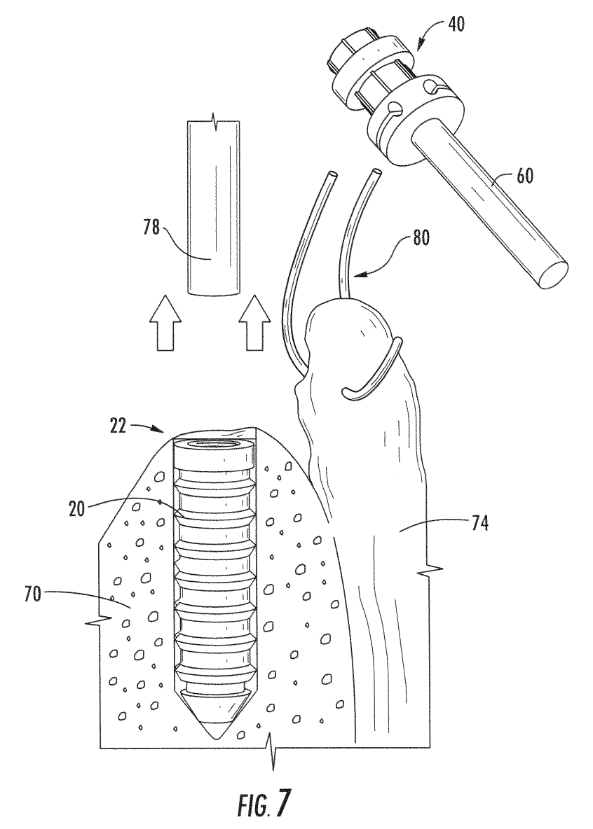

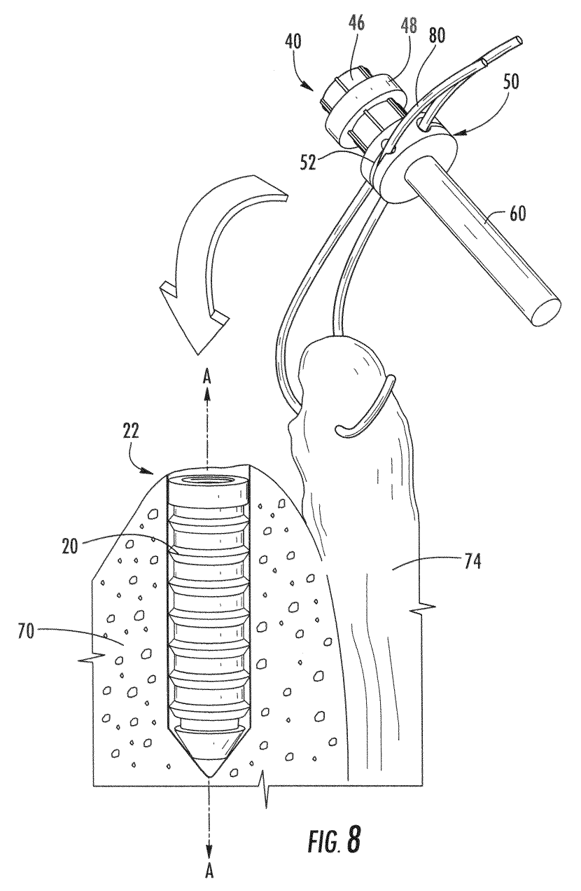

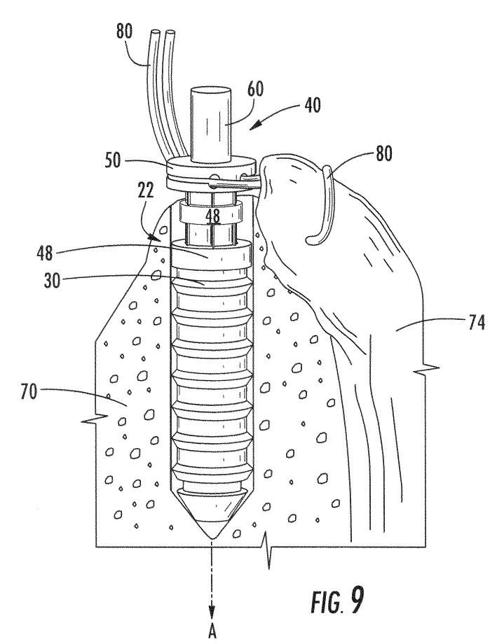

As shown in FIG. 7, once the anchor element 20 is positioned into the bone hole 72, the push rod 78 is removed from the anchor element 20, and the suture joiner element 40 is brought into position proximal to the suture 80. FIG. 8 shows suture 80 securely fed into the grooves 52 of the suture retaining element 50. The suture joiner element then is rotated into position to align the connection rod 46 along axis A and proximal to the distal end 22 of the bone anchor element 20. Using the connection rod 46, and as shown in FIG. 9, the suture joiner element 40 is pushed into position within the bone anchor element 20. Because the bone anchor element 20 is securely positioned within the bone 70, it acts as a stationary anvil against which the user can apply force, such that by applying downward force on the connection rod 46, it forces the flange 48 to fit within the anchor bore 30.

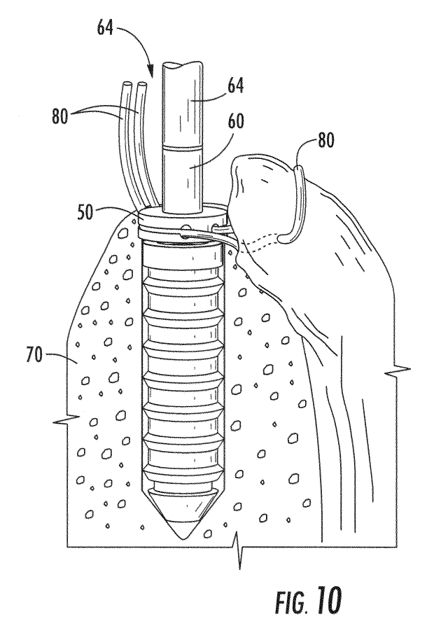

In a final step, and as shown in FIG. 10, a weld horn 64 is moved into position against the drive rod 60. Transmission of ultrasonic energy into the connection rod 60 causes the groove 52 to fuse to or around suture contained therewithin, thus essentially permanently securing the suture within the groove. In alternate embodiments, the groove 52 fuses closed around the suture, fuses the groove walls with the suture or partially closes the suture sufficiently to secure the suture. In alternative embodiments heat energy or physical energy is applied to the joiner element 50 sufficient to at least partially close the groove 52 around the suture therewithin.

In a preferred embodiment, and as shown in FIG. 10, a next step may include transmission of ultrasonic energy into the connection rod 60 or the anchor element 20 from the well horn 64. Such transmission causes vibration of the joiner element 40 relative to the anchor element 20, and in particular the relative movement of the energy directors (not shown in this FIG. 10) against the interfering portions of the stationary component, causes localized melting of the energy directors and the corresponding interfering portions of the stationary component to create weld regions. This welding step may not be necessary if the snap fit is sufficient to permanently secure the anchor element 20 with the suture joiner element 40.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description. All changes that come within the meaning and range of the equivalency of the claims are therefore intended to be embraced therein.

What is claimed:

1. A soft tissue fixation assembly for securing soft tissue to bone, the assembly comprising:
a bone anchor element extending along an anchor axis between a proximal end and a distal end and adapted for installation into a hole in a bone, including an anchor portion at the distal end;
a suture joiner element extending along a joiner axis between a proximal and a distal end having a suture retaining element at or near the proximal end thereof, the suture retaining element including a groove extending at least partially circumferentially thereabout for receiving suture;
wherein the anchor element and the joiner element each include joiner portions adapted for making engagement with the anchor axis and the joiner axis being substantially coaxial, and
wherein the suture joiner element is adapted to fixedly engage suture in the groove upon application of energy to the joiner element.

2. The soft tissue fixation assembly of claim 1, wherein the anchor portion includes a helical pattern of thread.

3. The soft tissue fixation assembly of claim 1, wherein the anchor portion includes a concentric ring pattern thread.

4. The soft tissue fixation assembly of claim 1, further comprising one or more energy directing members extending axially on the joiner portion of at least one of the anchor element and the joiner element for establishing a fusible interface therebetween, wherein the anchor element and the joiner element are adapted to be fused together in situ at the fusible interface upon application of energy to at least one of the anchor element and the joiner element.

5. The soft tissue fixation assembly of claim 1, wherein the bone anchor element further includes a drive portion at the proximal end thereof adapted to selectively receive a drive element for positioning the bone anchor element into the bone.

6. The soft tissue fixation assembly of claim 1, wherein the bone anchor element further includes an anchor bore extending along its axis from the proximal end thereof and the suture joiner element further includes a joiner connection rod extending along its axis from the distal end thereof, wherein the joiner connection rod is adapted to fit within the bore of the bone anchor element, whereby the anchor bore and the joiner connection rod form the joiner portions.

7. The soft tissue fixation assembly of claim 6, wherein the connection rod is adapted for directing applied energy to the energy directing members.

8. The soft tissue fixation assembly of claim 6, wherein the connection rod further comprises a circumferentially extending flange portion on at least a portion thereof, and the bore includes a circumferentially extending recess adapted to interfit with said flange for securely retaining the connection cod within the bone anchor element.

9. The soft tissue fixation assembly of claim 8, wherein the flange portion extends substantially around the circumference of a distal portion of the connection rod.

10. The soft tissue fixation assembly of claim 1, wherein the suture joiner element includes a drive portion at its proximal end adapted to selectively receive a drive element for positioning the suture joiner element into the bone anchor element.

11. The soft tissue fixation assembly of claim 1, wherein the suture retaining element includes two grooves, positioned end-to-end around at least partially about the circumference of the element, each groove for securely retaining suture therein.

12. The soft tissue fixation assembly of claim 1, wherein the suture joiner element is adapted to fuse around suture in the groove.

13. The soft tissue fixation assembly of claim 1, wherein the suture retaining element is substantially circular in shape radially extending about the axis or the joiner element.

14. A method of fixing soft tissue to a bone, comprising the steps of:
A. providing a soft tissue fixation assembly, the assembly including:
a bone anchor element extending along an anchor axis between a proximal end and a distal end and adapted for installation into a hole in a bones including an anchor portion at the distal end;
a suture joiner element extending along a joiner axis between a proximal and a distal end having a suture retaining element at or near the proximal end thereof the suture retaining element including a groove extending at least partially circumferentially thereabout for receiving suture;
wherein the anchor element and the joiner element each include joiner portions adapted for making engagement with the anchor axis and the joiner axis being substantially coaxial, and one or more energy directing members extend axially on the joiner portion of at least one of the anchor element and the joiner element for establishing a fusible interface therebetween, wherein the anchor element and the joiner element are adapted to be fused together in situ at the fusible interface upon application of energy to at least one of the anchor element and the joiner element; and
wherein the suture joiner element is adapted to fuse to or around suture in the groove upon application of energy to the joiner element;
B. installing the anchor element into a bone at a desired location;
C. positioning suture connected to soft tissue to be fixed, in the groove of the suture retaining element of the suture joiner element;
D. tensioning the sutures;
E. matingly engaging the suture joiner element and the bone anchor element; and
F. fusing the suture joiner element to or around the suture in the groove thereof.

15. The method of claim 14, wherein the energy applying step comprises:
transmitting energy to at least one of the suture joiner element and the bone anchor element to effect localized melting and fusing of interfacing portions of the suture joiner element and the bone anchor element arid fusing the suture joiner element to or around the suture in the groove.

16. The method of claim 14 wherein the applied energy is ultrasonic energy.

17. The method of claim 14 wherein the applied energy is heat.

18. The method of claim 14 wherein the steps are applied in sequence A, B, C, D, E and F.

19. The method of claim 14 wherein the steps are applied in sequence A, B, E, C, D and F.

20. The method of claim 14 wherein step B includes the sub-steps of:
(i) drilling a hole in the bone at the desired location;
(ii) inserting the anchor portion of the bond anchor element; and
(iii) driving the bone anchor element into said hole.

21. The method of claim 14 wherein the anchor portion of the bone anchor element is a self-drilling element, and wherein step B includes the sub-steps of:
(i) positioning the bone anchor portion at the desired location on the bone and (ii) driving the bone anchor into the bone at the desired location on the bone.

22. The method of claim 14, wherein step E further comprises applying energy to fuse the suture joiner element to the bone anchor element.
